(19) United States
(12) Patent Application Publication
(10) Pub. No.: US 2023/0062867 A1
(43) Pub. Date: Mar. 2, 2023

(54) WEARABLE DEVICE

(71) Applicant: DREEM, Paris (FR)

(72) Inventors: Martin HERRERA, Paris (FR); Gabriel OPPETIT, Paris (FR); Quentin SOULET DE BRUGIERE, Paris (FR)

(73) Assignee: DREEM, Paris (FR)

(21) Appl. No.: 17/770,911

(22) PCT Filed: Nov. 2, 2020
(86) PCT No.: PCT/FR2020/051976

§ 371 Date: Apr. 21, 2022

(30) Foreign Application Priority Data
Nov. 4, 2019 (FR) 19 12349

(51) Int. Cl. A61B 5/00; A61B 5/27

(52) U.S. Cl. CPC A61B 5/6804; A61B 5/27

(57) ABSTRACT

A wearable device including a garment portion including an inner textile layer, and an electronic portion including at least one electrode based on an electrically conductive elastomer, stitched to the inner textile layer and/or to a support layer attached to the inner textile layer. The garment portion is designed to at least partially surround a person's limb such that the inner textile layer and the at least one electrode are kept at least partially in contact with the person when the device is worn.

### TECHNICAL FIELD

This disclosure relates to the field of electronic devices and more specifically to wearable devices, i.e. electronic devices that can be worn as clothing or as clothing accessories, such as headbands, helmets, hats, watches, bracelets, belts, etc. The term “habitronic” is sometimes used.

### PRIOR ART

More and more electronic devices intended to be worn by a person during daily life make it possible to measure various parameters when worn, for example physiological parameters such as temperature or respiratory rate, or activity indicators such as a number of steps.

In parallel, it is known in the medical field to measure and record electrical signals from a person's body, using various devices. These electrical signals can be used to quantify or qualify brain activity or heart rate, for example. However, the medical devices are not designed for continuous wear in everyday life.

Typically, electroencephalography techniques use measurements of brain electrical activity which rely on one or more measurement electrodes placed on the scalp.

For the measurements to be representative and reproducible, the measurement conditions must be standardized. One known approach is the use of “wet” electrodes. These electrodes require the application of a conductive gel to the surface of the skin in order to ensure an electrical connection between the skin and the measurement electrode. However, the conductive gel used dries out over time, which limits the measurement acquisition time. In addition, the use of gel is uncomfortable for the person and is therefore difficult to adapt to everyday life. Finally, some people may be allergic to the conductive gel used.

Another approach is to press the electrode(s) firmly against the person's skin using mechanical devices. For example, the electrode(s) may be glued to the inside surface of a helmet that is strapped to the person. Wearing such a helmet is not very suitable for everyday life or even for a night of sleep.

It is therefore desirable to propose a device that makes it possible to reliably measure electrical signals coming from a person's body while being comfortable for the person to use, both during the person's daily activity and during sleep.

### SUMMARY

This disclosure improves the situation.

A wearable device is proposed, comprising:
a garment portion comprising an inner textile layer, and
an electronic portion comprising at least one electrode, based on electrically conductive elastomer, stitched to the inner textile layer and/or to a support layer attached to the inner textile layer,
   the garment portion being arranged so as to at least partially surround a person's limb such that the inner textile layer and the at least one electrode are kept at least partially in contact with the person when the device is worn.

As the inner textile layer and the at least one elastomer-based electrode are made of flexible materials, the device is comfortable for the person to wear. Provision may be made for the textile of the inner textile layer to be able to absorb moisture, typically perspiration, which provides increased comfort for the person.

In addition, the electrically conductive elastomer is a suitable material for the acquisition of electrical signals coming from the person's body. Indeed, the electrical signal produced for example by neurons is attenuated when passing through the skull, so the electric potential at the surface of the scalp is low. An electrode made of electrically conductive elastomer has sufficient sensitivity to detect and measure such electric potential in a manner enabling its analysis.

The elastomer-based electrode is stitched to the inner textile layer or stitched to a support layer attached to the inner textile layer. This provides both a reliable attachment and a flexibility enabling movements of the electrode, particularly in comparison to thermal bonding. Due to the fact that the electrode is stitched, it remains firmly attached to the garment portion while allowing the absorption of more deformation stresses without breaking than what bonding allows. Although stitching requires piercing the electrode, this does not have a substantial negative impact on the quality of the electrical signal measurement. Finally, the use of thread rather than an adhesive makes it possible to reduce the harmful ecological and health consequences of manufacturing the device and facilitates both its possible repair and its recycling at the end of its life.

Thus, due to the particular design of the features of the wearable device, and due in particular to the fact that the at least one electrode is stitched to the inner textile layer or to a support layer attached to the inner textile layer, the at least one electrode can be kept in contact with a person when the device is worn, regardless of the person's movements during daily activities or during sleep. It is thus possible to measure over time the electrical signals coming from the person's body in a reliable manner, without any application of gel being required.

In addition, even in the event that the wearable device is washed in the washing machine, the electrode can remain attached to the rest of the device, so the electrode remains able to measure the electrical signals coming from the person's body after washing in the washing machine and without the need to disassemble the electrode before washing.

The features discussed in the following paragraphs may optionally be implemented. They may be implemented independently of each other or in combination with each other:

In one embodiment, the garment portion is arranged so as to at least partially surround the person's head and such that at least one of the at least one electrode is kept at least partially against the person's forehead when the device is worn.

In one embodiment, the garment portion is arranged so as to at least partially surround the person's head such that at least one of the at least one electrode is kept at least partially against the person's occiput when the device is worn.

During sleep, different electrical phenomena are generated by different areas of the brain.

The forehead and occiput are particularly suitable areas for the measurement of usable signals, in particular for classifying the wearer's sleep stages. Such areas may thus be preferred for positioning the electrodes on the headband. Since these areas are preferred, different embodiments make it possible to optimize the person's comfort in particular.

In the case where the electrode is stitched to a support layer attached to the inner textile layer, the method of attaching the support layer to the inner textile layer may also be stitching. For example, it is possible, by means of a single stitching through the electrode, support layer, and inner textile layer, to attach the electrode to the support layer and to the inner textile layer (a single thread and a shared stitching operation). Alternatively, the support layer may be attached to the inner textile layer by means of bonding, for example thermal bonding.

In one embodiment, the support layer is made of textile material.

Thus, the garment portion may be formed of the support layer made of textile material, the inner textile layer, and possibly additional textile layers, forming a set of superimposed layers of textile material. A garment portion comprising such a set of superimposed layers is both flexible and slightly compressible, which allows the inner textile layer to conform to the shape of the limb at least partially surrounded when the wearable device is worn, which is particularly comfortable for the person.

In one embodiment, the at least one electrode is produced from carbon-doped silicone elastomer.

After comparative tests between different elastomers, the inventors noticed that, although other materials also allow the acquisition of an electrical signal coming from a person's body, an electrode based on carbon-doped silicone elastomer is particularly sensitive, such that the output signal from such an electrode allows detecting and measuring a potential, or by extension a variation in potential over time, of particularly low amplitude. The accuracy of the signal measured by such an electrode can thus be equivalent to that of a signal from a “wet” electrode (involving the use of gel).

In one embodiment, the at least one electrode is functional at any temperature between −10° C. and 50° C. Preferably, the at least one electrode is functional at any temperature within a wider temperature range, i.e. between −55° C. and 200° C.

Thus, the at least one electrode is functional when the wearable device is worn in virtually any humanly acceptable environment and remains functional even after exposure to extreme temperature conditions.

In one embodiment, the electronic portion further comprises an electronic unit mechanically and electrically connected to the at least one electrode in a removable manner.

Such an electronic unit may comprise a processing circuit, which may include for example a processor connected to a memory and to at least one interface for communicating with the at least one electrode, the processing circuit being configured to carry out the steps of a method for acquiring and processing an electrical signal coming from the person's body.

In one embodiment, the electrode comprises at least one groove in which a cable is mounted. The electrode is stitched to the support layer by at least one thread forming a plurality of loops, and a loop of said plurality of loops straddles the groove so as to hold the cable therein. We distinguish here the electrical conductor from the mechanical link of the stitching by the respective use of the terms “cables” and “thread”.

The cable makes it possible to transmit an electrical signal captured by the at least one electrode to a processing circuit, for example integrated into said electronic unit.

The groove serves to guide the cable, in order to hold the cable in a predetermined position when stitching the electrode to the support layer. However, a groove in the electrode is not essential for guiding the cable into the predetermined position. For example, the garment portion may comprise a groove or hole intended to receive the cable connected to the electrode, this groove or hole then serving to guide the cable and maintain the cable in a predetermined position including along the surface of the electrode.

When stitching the electrode to the support layer by means of thread, it is preferable to ensure that the thread, and especially the stitching tools, do not damage the cable. Locking the cable in a predetermined position makes it possible to base the stitching on this predetermined position, so that the thread does not damage the cable but instead forms a loop spanning the predetermined position. Thus, after stitching, the thread holds the cable in the predetermined position, within the groove. The position of the stitching may be defined relative to a reference marker and a predetermined position of the cable. Such a positional reference marker may, for example, take the form of a marking on the surface of the electrode or an edge of the electrode.

Attachment of the cables by thread makes it possible to achieve, in one operation, both the assembly of the electrode to the body of the device and that of the cabling. It is then unnecessary to add dots of glue to fix the cables, with the same environmental and health benefits.

In one embodiment, a groove is provided on a surface of the electrode arranged to be held against the person's skin when the device is worn. In addition, the thread is arranged in the groove. This particular arrangement thus prevents the stitching from irritating the person's skin. Such an arrangement is particularly suitable in the case of an occipital electrode.

In one embodiment, the electronic portion comprises a plurality of electrodes based on electrically conductive elastomer, each electrode being stitched to the inner textile layer or to a support layer attached to the inner textile layer. The garment portion is arranged so as to at least partially surround the person's head such that two groups of electrode(s) of the plurality of electrodes are kept at least partially against the person's forehead and occiput respectively when the device is worn.

For example, it is possible to provide at least four prefrontal electrodes and at least two occipital electrodes, arranged according to the international 10/20 system of electrode placement, in order to measure brain activity. The numbers 10 and 20 denote the fact that the distance between two adjacent electrodes is from 10% to 20% of the total distance either between the front and back of the head or between the left side and right side of the skull.

In one embodiment, the garment portion further comprises an outer textile layer opposite to the inner textile layer. The support layer is sandwiched between the inner textile layer and the outer textile layer.

In this arrangement, the support layer is insulated from the person's skin by the inner textile layer when the device is worn. In addition, the support layer is insulated by the inner textile layer and by the outer textile layer when the device is held in the hand. This arrangement is particularly advantageous when the support layer is formed of a material that is rigid and/or unpleasant to the touch, or formed of a material that is less wear-resistant than a textile.

In one embodiment, the inner textile layer comprises an outer surface facing the support layer and an inner surface opposite to the outer surface of the inner textile layer. The at least one electrode is arranged against at least part of the inner surface of the inner textile layer.

This arrangement is of particular interest in the case of occipital electrodes. Indeed, to capture the electrical signals, it is preferable that an occipital electrode be in contact with the person's skin and not solely with the person's hair. To achieve this objective, it is advantageous for the occipital electrode to be arranged between at least part of the inner textile layer and the person's skin when the device is worn.

Another arrangement may be preferable, particularly in the case of prefrontal electrodes. Indeed, it may be advantageous to arrange the electrode so that the contact surface of the electrode forms, with the inner textile portion, a substantially smooth, homogeneous surface. This increases the person's comfort while allowing good acquisition of an electrical signal. This objective may be achieved by arranging the prefrontal electrode partly between the inner textile layer and a support layer so that the prefrontal electrode comprises a contact portion arranged flush with or slightly projecting from the inner textile layer.

### BRIEF DESCRIPTION OF DRAWINGS

Other features, details, and advantages will become apparent upon reading the detailed description below and analyzing the appended drawings, in which:

FIG. 1

FIG. 1 shows a sectional view of a first part of a wearable device according to one embodiment.

FIG. 2

FIG. 2 shows a sectional view of a second part of the wearable device of FIG. 1.

FIG. 3

FIG. 3 shows a view of an electrode of a wearable device according to one embodiment.

### DESCRIPTION OF EMBODIMENTS

For the most part, the drawings and the description below contain elements that are certain in nature. Therefore not only may they serve to provide a better understanding of this disclosure, but where appropriate they may also contribute to its definition.

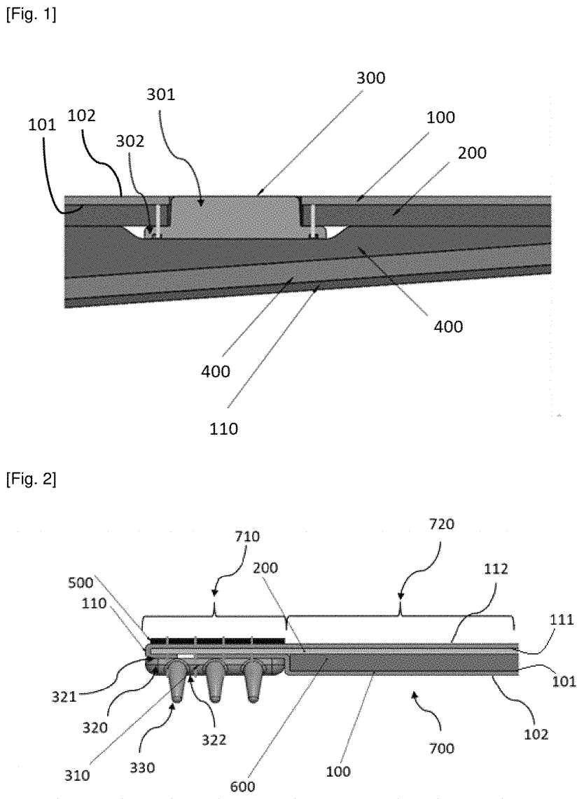

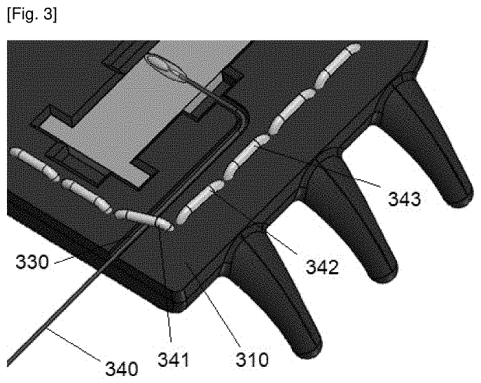

Reference is now made to FIGS. 1 and 2, which represent two sectional views, respectively of a first part and of a second part of a wearable device according to one embodiment.

According to this embodiment, the wearable device is a headband in which the first part as represented in FIG. 1 is a front part.

The front part comprises a garment portion and an electronic portion.

The garment portion comprises:
an inner textile layer 100 configured to be kept in contact with a person's skin when the wearable device is worn by the person;
an outer textile layer 110 opposite to the inner textile layer 100; and
a support layer 200, or backing layer, interposed between the inner textile layer 100 and the outer textile layer 110.

The electronic portion comprises at least one electrode 300, here a prefrontal electrode, based on electrically conductive elastomer, stitched to the support layer 200.

Although in this example the electrode is stitched to the support layer 200, such a support layer 200 may be absent. The support layer 200 here makes it possible to reinforce the garment portion.

Similarly, the outer textile layer 110 is provided in this example to form, with the inner textile layer 100, two outer layers of a set of superimposed layers. Alternatively, the outer textile layer 110 is absent.

The outer textile layer 110 may be a textile piece that is distinct from the inner textile layer 100. Alternatively, the outer textile layer 110 and the inner textile layer 100 may be the same textile piece, folded onto itself to form the two aforementioned layers.

Alternatively, the electrode 300 may be stitched to another element of the garment portion, for example to a garment portion consisting solely of the inner textile layer 100.

Alternatively, the electrode 300 could be stitched to a garment portion comprising the inner textile layer 100 and at least one support layer 200, by a thread passing through both the inner textile layer 100 and said at least one support layer 200.

Alternatively, the electronic portion could comprise a plurality of electrodes 300, each electrode 300 being stitched to the inner textile layer 100 and/or to a support layer 200 dedicated to said electrode 300 and/or to a support layer 200 common to a plurality of electrodes 300.

The electrode 300 and the inner textile layer 100 are mutually arranged so that, when the device is worn by a person, at least part of the electrode 300 is kept in contact with the person, and more particularly, in the example shown, with the person's forehead.

In one embodiment, the inner textile layer 100 comprises an outer surface 101 facing the support layer 200 and an inner surface 102, opposite to the outer surface 101 of the inner textile layer 100, and configured to be in contact with a person's skin when the wearable device is worn by the person.

A hole may be made through the inner textile layer 100, the hole leading from the outer surface 101 of the inner textile layer 100 to the inner surface 102 of the inner textile layer 100.

In one embodiment, at least one sensor may be mounted in the hole. For example, such a sensor may be a pulse oximeter arranged so as to face the person's forehead when the device is worn, thus also making it possible to evaluate the person's blood oxygen saturation level.

In one embodiment, the electrode 300 may be mounted in the hole and arranged so as to comprise a contact portion 301 placed flush with or projecting from the inner surface 102 of the inner textile layer 100, so as to be held against the person when the device is worn, and an attachment portion 302 extending to the support layer 200, the attachment portion 302 being stitched to the support layer 200 by at least one thread passing through at least the attachment portion 302 and the support layer 200.

By this particular arrangement, the attachment portion 302, the support layer 200, and the stitching (the path of the thread) are internal to the device. The stitching is thus protected against wear.

In addition, as only the contact portion 301 is flush with or projecting from the inner surface 102 of the inner textile layer 100, the attachment portion 302 is inaccessible from outside the device, so that there is little risk of the electrode 300 being torn off.

In one embodiment, the hole is also made through the support layer 200. The attachment portion 302 is placed between the support layer 200 and the outer textile layer 110.

By this particular arrangement, the robustness of the attachment of the electrode 300 is reinforced. Indeed, the attachment portion 302 of the electrode bears against the periphery of the hole in the support layer 200 on the inner side, thus preventing its accidental extraction.

In one embodiment, at least one layer of foam 400 is also arranged between the support layer 200 and the at least one electrode 300 on the one hand, and the outer textile layer 110 on the other hand. In the embodiment represented in FIG. 1, two layers of foam 400 are superimposed.

When the device is worn by a person, such a layer of foam, being flexible and easily compressible, easily conforms to the person's morphology and contributes to holding the device in place as well as to providing the person with a sensation of comfort. In addition, the presence of foam facilitates adapting to different morphologies by its reversible deformation and elastic effect.

According to one embodiment, the wearable device is a headband in which the second part as represented in FIG. 2 is an occipital part.

According to this embodiment, the wearable device comprises, in the occipital part as represented in FIG. 2, a garment portion including the inner textile layer 100, the outer textile layer 110, and the support layer 200. The inner textile layer 100 is configured, in the occipital part, to be kept in contact with a person's skin when the wearable device is worn by the person. The inner textile layer 100 optionally forms, with the outer textile layer 110 opposite to the inner textile layer 100 and with the support layer 200 interposed between the inner textile layer 100 and the outer textile layer 110, a set of superimposed layers 700.

According to the embodiment represented in FIG. 2, the wearable device further comprises, on the occipital part:
an electronic portion comprising at least one electrode 310, here an occipital electrode, based on electrically conductive elastomer, stitched to the inner textile layer 100, or to the set of superimposed layers 700, by at least one thread passing through the electrode 310 and, respectively, the inner textile layer 100 or the set of superimposed layers 700.

In one embodiment, the at least one electrode 310 comprises a base 320 stitched to the set of superposed layers 700 by said at least one thread passing through the set of superimposed layers 700, the base 320 comprising an outer surface 321 facing the inner textile layer 100 and an inner surface 322 opposite to the outer surface 321 of the base 320, and at least one tab 330 projecting from the inner surface 322 of the base 320.

By this particular arrangement, when the device is worn by a person, the electrode 310 is positioned entirely between the garment portion and the person.

In addition, the particular shape of the electrode is such that when the device is worn by a person, the at least one tab passes through the person's hair and is in contact with the person's skin, enabling good acquisition of an electrical signal coming from the person's body.

This particular arrangement associated with this particular form of electrode makes it possible to optimize the ratio between the weight of the electrode 310 and the surface area of the electrode 310 likely to come into contact with the person, relative to an embodiment in which the inner textile layer 100 is interposed between the person and at least part of the electrode 310 when the device is worn.

Thus, the acquisition by the electrode 310 of an electrical signal coming from the person's body is improved, while minimizing the weight of the electrode 310. This contributes to minimizing both the total weight of the device and the weight distribution within the device, therefore contributes to a comfortable and balanced wearing of the device.

In one embodiment, each layer of the set of superimposed layers 700 is of substantially uniform thickness. The set of superimposed layers 700 comprises a first part 710 in which the support layer 200 is directly attached to the inner textile layer 100, and a second part 720 in the extension of the first part 710. The second part 720 comprises a spacing layer 600 of a thickness substantially equal to the base 310 of the at least one electrode 300, arranged between the support layer 200 and the inner textile layer 100. The base 310 of the at least one electrode 300 extends only along the first part 710 of the set of superposed layers 700, so that the assembly consisting of the base 310 of the at least one electrode 300, the first part 710 of the set of superposed layers 700, and the second part 720 of the set of superposed layers 700, are of substantially uniform thickness along the device.

At least one securing layer 500 may be provided. An example of a securing layer 500 is a layer including an adhesive material, hooks, and/or textile loops.

According to this example, such a securing layer 500 is arranged against the outer surface 112 of the outer textile layer 110 or against the inner surface 102 of the inner textile layer 100. Thus the securing layer 500 makes it possible to maintain the garment portion mechanically in a position where it at least partially surrounds a person's limb.

Another example of a securing layer 500 may be a layer including an elastic material. According to this example, such a securing layer 500 forms one layer of the set of superimposed layers 700. For example, such a securing layer 500 may be interposed between the layers forming the second part 720 of the set of superimposed layers 700.

Such a securing layer 500 may be a layer dedicated to holding the garment portion in a position where it at least partially surrounds a person's limb. Alternatively, such a securing layer 500 may have an additional function. For example, the inner textile layer 100 and/or the support layer 200 may include an elastic material and thus also constitute securing layers 500.

Reference is now made to FIG. 3, which represents a partial view of an electrode of a wearable device according to one embodiment.

The electrode 300, 310 may be electrically connected to an electronic processing circuit (not shown) via a cable 340 enabling the electrode 300, 310 to transmit one or more measurements captured over time, to a communication interface of the electronic processing circuit.

An example of such an electronic processing circuit comprises a processor connected to a memory and to at least one communication interface for at least one electrode 300, 310. The processing circuit is configured to carry out the steps of a method for acquiring, and optionally processing, at least one electrical signal captured by said at least one electrode.

In one embodiment, such an electronic processing circuit may be integrated into a unit connected mechanically and electrically in a removable manner, for example via the cable 340, to the electrode 300, 310.

In the example described here, the electrode 300, 310 comprises, formed in a surface of said electrode, a groove 330 arranged to at least partially house and guide the cable 340 along said surface.

The cable 340 is thus held/guided in a predetermined position.

The method of manufacturing the wearable device comprises in particular a step of stitching the electrode 300, 310 to the inner textile layer 100 or to a support layer 200 or to a set of superimposed layers 700 comprising at least the inner textile layer 100 and the support layer 200, by a thread passing through said layer or layers 100, 200.

The thread thus forms a plurality of loops 341, 342, 343 connecting the electrode 300, 310 to said layer(s) 100, 200.

During this stitching step, if the electrode 300, 310 was previously connected to a cable 340, it is necessary to ensure that the loops 341, 342, 343 and the stitching tools do not damage the cable.

To overcome this problem, one possibility is to rely on a positional reference marker, provided for example on a surface or an edge of the electrode 300, 310 for determining a planned position of said loops 341, 342, 343, then implementing the stitching step on the basis of the positions thus determined.

If the electrode 300, 310 comprises, in a surface of said electrode 300, 310, a groove 330 serving to guide the cable 340 along said surface, then the groove 330 is an example of such a positional reference marker.

It can thus be provided that the position of a loop 341 is determined so that said loop 341 spans the groove 330, further contributing to maintaining the cable 340 in a predetermined position, here within the groove 330.

This disclosure is not limited to the examples described above in support of FIGS. 1 to 3 presented solely as examples for the purpose of facilitating an understanding of the invention, but encompasses all variants conceivable to a person skilled in the art within the context of the protection sought.

1-10. (canceled)

11. A wearable device comprising:
a garment portion comprising an inner textile layer, and
an electronic portion comprising at least one electrode, based on electrically conductive elastomer, stitched to the inner textile layer and/or to a support layer attached to the inner textile layer,
the garment portion being arranged so as to at least partially surround a person's limb such that the inner textile layer and the at least one electrode are kept at least partially in contact with the person when the device is worn.

12. The wearable device according to claim 11, comprising a support layer made of textile material, attached to the inner textile layer.

13. The wearable device according to claim 11, wherein the at least one electrode is produced from a carbon-doped silicone elastomer.

14. The wearable device according to claim 11, wherein the at least one electrode is functional at any temperature between −10° C. and 50° C.

15. The wearable device according to claim 11, wherein the electronic portion further comprises an electronic unit mechanically and electrically connected to the at least one electrode in a removable manner.

16. The wearable device according to claim 11, wherein:
at least one of the at least one electrode comprises at least one groove in which a cable is mounted;
the at least one of the at least one electrode is stitched by at least one thread forming a plurality of loops, and
a loop of said plurality of loops straddles the groove so as to hold the cable therein.

17. The wearable device according to claim 11, the garment portion being arranged so as to at least partially surround the person's head and such that at least one of the at least one electrode is kept at least partially against the person's forehead when the device is worn.

18. The wearable device according to claim 11, wherein the garment portion is arranged so as to at least partially surround the person's head such that at least one of the at least one electrode is kept at least partially against the person's occiput when the device is worn.

19. The wearable device according to claim 11, wherein:
the garment portion further comprises an outer textile layer opposite to the inner textile layer, and
the support layer is sandwiched between the inner textile layer and the outer textile layer.

20. The wearable device according to claim 19, wherein:
the inner textile layer comprises an outer surface facing the support layer and an inner surface opposite to the outer surface of the inner textile layer, and
the at least one electrode is arranged against at least part of the inner surface of the inner textile layer.
